(19) United States
(12) Patent Application Publication
(10) Pub. No.: US 2004/0092482 A1
(43) Pub. Date: May 13, 2004

(54) Hydroxy acids based delivery systems for skin resurfacing and anti-aging compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/290,933

(22) Filed: Nov. 7, 2002

(51) Int. Cl. A61K 31/728; A61K 31/7008; A61K 31/4172; A61K 31/375; A61K 31/19; A61K 31/205

(52) U.S. Cl. 514/62; 514/54; 514/400; 514/557; 514/474; 514/102; 514/554

(57) ABSTRACT

This invention relates to in-situ preparation of the derivatives of various hydroxy acids (HA), such as α-(Alpha) Hydroxy Acids (AHA), β-(Beta) Hydroxy Acids (BHA), and Poly-Hydroxy Acids (PHA) with certain skin beneficial organic hetero-atom bases and their application in skin resurfacing (exfoliation), and in the synergistic treatment and regulation of topical disorders of skin such as skin aging, wrinkles, acne, rosacea, age-spots, canker sores, striae distensae (stretch marks), pimples, skin redness, and dry skin conditions of cracking, flaking, and scaling. Most HA derivatives produced by the in-situ method do not cause skin irritation and skin redness effects that are commonly experienced with AHA and BHA, yet there is no loss of their skin beneficial effects. These compositions can be traditional water and oil emulsions, liposomes, suspensions, colloids, solutions, masks, muds, serums, sprays, gels, lotions, creams, cleansers, and anhydrous systems, thus offering a wide choice of formulations to meet their consumer appeal and acceptance requirements.

### BACKGROUND OF THE INVENTION

This invention relates to in-situ preparation of the derivatives of various hydroxy acids (henceforth called “HA”), such as α-(Alpha) Hydroxy Acids (henceforth called “AHA”), β-(Beta) Hydroxy Acids (henceforth called “BHA”), and Poly-Hydroxy Acids (henceforth called “PHA”) with certain skin beneficial organic hetero-atom bases and their application in skin resurfacing (exfoliation), and in the synergistic treatment and regulation of topical disorders of skin such as wrinkles, acne, rosacea, age-spots, canker sores, striae distensae (stretch marks), pimples, skin redness, and dry skin conditions of cracking, flaking, and scaling and skin aging. The in-situ method also permits the simple preparation of certain novel derivatives of such hydroxy acids from commonly available ingredients. Most HA derivatives produced by the in-situ method do not cause skin irritation and skin redness effects that are commonly experienced with AHA and BHA, yet there is no loss of their skin beneficial effects. The compositions that contain such derivatives of HA can be traditional water and oil emulsions, suspensions, liposomes, colloids, solutions, or anhydrous systems.

HA's have been proven to deliver cosmetic benefits, such as improvement in the appearance of proto-damaged or naturally aged skin, skin lightening, treatment of age spots, etc. Glycolic acid has been used in many cosmetic formulations for improved skin appearance. There are two main theories on how glycolic acid works. The first theory proposes that the glycolic acid produces a mild sub clinical irritation which stimulates the epidermis to produce fresh skin, while the second theory proposes that glycolic acid weakens the intercellular bonding of the corneocytes in a manner similar to both water and retinoids. Unfortunately, little objective data regarding the effectiveness of alpha-hydroxy acid has been published thereby leaving the industry to rely on anecdotal information, which is difficult to quantify. It is quite clear that many of the topical cosmetics incorporating glycolic acid or other alpha-hydroxy acids have insufficient concentrations to accomplish their objectives. The human skin is comprised of two principal components, the avascular epidermis and the underlying vascular dermis. The epidermis consists of four layers: the stratum corneum, stratum granulosum, stratum spinosum and stratum basale. The dermis mainly consists of collagen, elastin fibers and ground substances including glycosaminoglycan. There are two forms of skin aging: intrinsic aging, also known as chronological aging and extrinsic aging, also known as photo aging. The aging process normally involves the dermis. Intrinsic aging is a degenerative process attributed to declining physiologic functions and capacities. Extrinsic aging is caused by external factors such as sunlight, radiation, air pollution, etc. AHA's have been used topically in the prior art on keratinization (epidermal layer) where the effects are clinically detectable by the formation of a new stratum corneum. AHA's also have dermal effects. Topical applications of AHA's have caused increased amounts of mucopolysaccharides and collagen and increased skin thickness without detectable inflammation.

The benefits of the AHA have caused them to be incorporated into cosmetic products for purposes such as cleansing, conditioning, dry skin etc. AHA's are categorized as nontoxic and have been used as skin desquamation agents, especially in routine use for acne, wrinkles, photo aged skin and pigmented disorders. Mandelic acid, another AHA, has been claimed by Yu et al. (U.S. Pat. No. 5,677,339) in a topical composition for retarding the effect of aging on human facial skin, and the same authors report (U.S. Pat. No. 5,654,336) the skin wrinkles reduction by AHA. Glycolic and lactic acids have been claimed in pimples and skin redness reduction compositions by Slavtcheff et al. (U.S. Pat. No. 5,614,201 and 5,482,710). Alliger (U.S. Pat. No. 5,516,799) describe the use of glycolic acid for treating small mouth ulcers. Shaffer et al. (U.S. Pat. No. 5,760,079) describe hydroxy acids for treating striae distensae (stretch marks). Perricone (U.S. Pat. No. 6,417,226) has claimed Hydroxytetronic acid in a skin whitening composition. Other AHA's have shown skin-whitening effects, as mentioned by Zhang et al. (U.S. patent application 20020106384). Goodman (U.S. patent application 20020155180) reports the use of AHA in treating acne and hirsutism. U.S. Pat. No. 5,861,432 to Sklar describes the use of glycolic acid in an acne treatment formulation. U.S. Pat. No. 5,705,170 (Kong et al.) discloses an herbal cellulite treatment, which may contain AHA. U.S. Pat. No. 5,053,222 (Takasu et al.) discloses a hair composition for dandruff treatment, which may contain optional ingredients such as AHA. U.S. Pat. No. 3,897,537 describes AHA useful in the treatment of ichthyosiform dermatoses. U.S. Pat. Nos. 3,984,566; 3,988,470, and 3,920,835 describe AHA treatments for skin lesions, which accompany disturbed keratinization that includes dandruff, acne, hyperkeratosis and calluses. U.S. Pat. No. 4,105,733 describes dry skin conditions of cracking, flaking, and scaling with AHA.

There is no doubt that alpha hydroxy acids (AHA), beta hydroxy acids (BHA), poly hydroxy acids (PHA) and related compounds are therapeutically effective for topical treatment of various cosmetic conditions and dermatological disorders including dry skin, acne, dandruff, keratoses, age spots, wrinkles and disturbed keratinization. However, the compositions containing these acids may irritate human skin on repeated topical applications due to lower pH of the formulations, as discussed in detail by Santhanam et al. (U.S. patent application 20020009508 and U.S. Pat. No. 6,277,881), Weinkauf et al. (U.S. Pat. No. 6,022,896) Habif et al. (U.S. Pat. No. 5,989,572), Duffy (U.S. Pat. No. 5,516,793), and Groh (U.S. Pat. No. 5,863,943). See also Kligman et al. (J. Geriatr. Dermatol. 1997; 5(3):128-131). The irritation may range from a sensation of tingling, itching and burning to clinical signs of redness and peeling. Causes for such irritation may arise from the following: Upper layers of normal skin have a pH of 4.2 to 5.6, but the compositions containing most alpha hydroxy acids or alpha ketoacids have pH values of less than 3.0. For example, a topical formulation containing 7.6% (1M) glycolic acid has a pH of 1.9, and a composition containing 9% (1M) lactic acid has the same pH of 1.9. These compositions of lower pH on repeated topical applications can cause a drastic pH decrease in the stratum corneum of human skin, and provoke disturbances in intercorneocyte bondings resulting in adverse skin reactions, especially to some individuals with sensitive skin. Moreover, with today's state of the art it is still very difficult to formulate a lotion, cream or ointment emulsion which contains a free acid form of the alpha hydroxyacid, and which is physically stable as a commercial product for cosmetic or pharmaceutical use. For example, Groh (U.S. Pat. No. 5,683,943) reports the use of a combination of a glycol and a quaternary ammonium surfactant to stabilize certain skin conditioner AHA compositions. The use of such surfactants may not be desirable in certain cosmetic applications, such as skin lotion, creams, paste, gel, serum, and such. Bimczok et al. (U.S. Pat. No. 5,961,999) reports the use of betaine esters in AHA compositions to provide skin compatibility. This is again very limited in application, as such betains act as surfactants and they can destabilize most skin lotion, cream, gel, and paste compositions. Yu et al. (U.S. Pat. No. 5,690,967 and 5,681,853) report methods for improving topical delivery of AHA by combining such acids with certain amphoteric or pseudoamphoteric ingredients, such as amino acids and peptides. However, such amphoteric ingredients usually have a free carboxyl group in their molecules, and under certain conditions of the manufacture of such compositions those carboxyl groups may get ionized and separate from their combination with AHA, thus causing product instability problems. Additionally, such amphoteric or pseudoamphoteric ingredients appear only to increase the pH of such compositions, and they do not appear to have any synergistic beneficial effect on skin. Moreover, many such amphoteric ingredients are not soluble in organic solvents commonly used in cosmetic compositions for the preparation of anhydrous systems that contain certain HA. U.S. Pat. Nos. 4,363,815; 4.380,549, and 5,091171 (Yu et al.) claim the combination of AHA's with certain amines, such as ammonium hydroxide, organic primary, secondary or tertiary amines, such as alkyl amines, alkanolamines, diamines, dialkyl amines, dialkanolamines, dialkylalkanolamines, and alkyl dialkanolamines wherein the alkyl or alkanol substituent has from 1-to-8 carbon atoms, methylamine, ethylamine, monoethanolamine, monoisopropanol amine, ethylene-diamine, 1,2-diaminopropane, dimethylamine, diethylamine, diethanolamine, diisopropanolamine, N-methylethanolamine, N-ethylethanolamine, triethylamine, triethanolamine, N-methyldiethanolamine, and triisopropylamine. However, the use of such strongly alkaline amines, resulted in the increase of the pH of such AHA, thus resulting in their much-lowered efficacy, as proclaimed in more recent references cited above. Moreover, many of such amines have strong, objectionable odor and hence not suitable for cosmetic compositions although they may be acceptable for certain pharmaceutical applications of AHA.

When a formulation containing an alpha hydroxyacid or alpha ketoacid is reacted equimolarly or equinormally with a metallic alkali such as sodium hydroxide or potassium hydroxide the composition becomes therapeutically ineffective. The reasons for such loss of therapeutic effects are believed to be as follows: The intact skin of humans is a very effective barrier to many natural and synthetic substances. Cosmetic and pharmaceutical agents may be pharmacologically effective by oral or other systematic administration, but many of them are much less or totally ineffective on topical application to the skin. Topical effectiveness of a pharmaceutical agent depends on two major factors; (a) bioavailability of the active ingredient in the topical preparation and (b) percutaneous absorption, penetration and distribution of the active ingredient to the target site in the skin. For example, a topical preparation containing 5% salicylic acid is therapeutically effective as a keratolytic, but that containing 5% sodium salicylate is not an effective product. The reason for such difference is that salicylic acid is in bioavailable form and can penetrate the stratum corneum, but sodium salicylate is not in bioavailable form for this specific skin beneficial function and cannot penetrate the stratum corneum of the skin. In the case of alpha hydroxy acids, a topical preparation containing 5% glycolic acid is therapeutically effective for dry skin, but that containing 5% sodium glycolate is not effective. The same is true in case of 5% lactic acid versus 5% sodium lactate. The reason for such difference is that both glycolic acid and lactic acid are in bioavailable forms and can readily penetrate the stratum corneum, but sodium glycolate and sodium lactate are not in bioavailable forms for the intended specific skin beneficial functions and cannot penetrate the stratum corneum of the skin. When a formulation containing an alpha hydroxyacid or alpha ketoacid is reacted equimolarly or equinormally with ammonium hydroxide or an organic base of smaller molecule the composition still shows some therapeutic effects for certain cosmetic conditions such as dry skin, but the composition has lost most of its potency for other dermatological disorders such as wrinkles, keratoses, age spots and skin changes associated with aging.

A number of inventions have been reported to overcome the skin irritation problems of AHA and BHA, and still maintain their skin beneficial efficacy. Santhanam et al. (U.S. patent application 20020009508) report the use of Echinacea extract as an anti-irritant to combat the skin irritation of certain HA. Habif et al. (U.S. Pat. No. 5,989,572) report the use of borage seed oil as an anti-irritant in HA compositions. Weinkauf et al. (U.S. Pat. No. 6,022,896) report the use of petroselinic acid as an anti-irritant for compositions that contain AHA. Santhanam et al. (U.S. Pat. No. 6,277,881) report the application of turmeric extract as an anti-irritant for AHA formulations. Duffy (U.S. Pat. No. 5,516,793) reports the use of ascorbic acid to reduce the irritation of AHA and BHA in topical preparations. Merianos (U.S. Pat. No. 5,728,390) reports the use of polyvinylpyrrolidone for minimizing the skin irritation effect of AHA. As is evident from the claims in the above mentioned prior art, the above methods are all very limited in their application, as they relate to the use of specific single ingredient that may not be acceptable in certain topical compositions that contain high levels of HA. Moreover, these anti-irritant ingredients do not appear to have any other synergistic beneficial effect on skin.

In a surprising discovery, the present invention reports the preparation of certain derivatives of AHA, BHA, and PHA with hetero-atom organic bases that do not have the loss of efficacy observed with the derivatives of such acids with ammonium hydroxide or organic bases of smaller molecule, as reported above. Moreover, the pH of such derivatives of AHA, BHA, and PHA with hetero-atom organic bases is typically higher than 3 and hence more compatible with skin's own pH range thus causing much less or no skin irritation. Additionally, such derivatives provide a synergistic combination of skin treatment benefits of both the acid and the hetero-atom base moieties of such derivatives. Finally, a great variety of such hetero-atom bases can be used for such synergistic combinations, thus providing a wider choice of formulation possibilities.

### OBJECTS OF THE INVENTION

This invention relates to in-situ preparation of the derivatives of various hydroxy acids (henceforth called “HA”), such as α-(Alpha) Hydroxy Acids (henceforth called “AHA”), β-(Beta) Hydroxy Acids (henceforth called “BHA”), and Poly-Hydroxy Acids (henceforth called “PHA”) with certain skin beneficial organic hetero-atom bases and their application in the synergistic treatment and regulation of topical disorders of skin such as skin aging, wrinkles, acne, rosacea, age-spots, canker sores, striae distensae (stretch marks), pimples, skin redness, and dry skin conditions of cracking, flaking, and scaling.

This invention also relates to compositions that include derivatives of HA with skin beneficial organic heteroatom bases that are prepared in-situ from the combination of HA, AHA, BHA, and PHA with such organic bases.

In a further respect, the invention relates to derivatives of HA with organic heteroatom bases that provide a combination of the skin beneficial properties of HA and organic base ingredients thus combined and additionally provide synergistic benefits. For example, glucosamine mandelate is made by the combination of glucosamine and mandelic acid. Glucosamine mandelate thus has the combination benefits, such as collagen synthesis enhancement by glucosamine, and skin rejuvenating property of mandelic acid. The absorption and penetration of glucosamine mandelate is more enhanced than the absorption of either glucosamine or mandelic acid, if used alone. Thus, glucosamine mandelate is more effective in wrinkle reduction due to its better synergistic absorption into the skin and its enhanced synergistic activation of collagen synthesis in the epidermal layers of skin, thus reducing the wrinkles.

In a further respect, this invention relates to in-situ preparation of novel derivatives of HA with skin beneficial organic heteroatom bases that can be made either in anhydrous systems, solutions, colloids, liposomes, or traditional water and oil emulsion systems, thus offering a wide choice of delivery systems.

### BRIEF DESCRIPTION OF THE INVENTION

I have discovered a simple in-situ preparation of the derivatives of HA with certain hetero-atom organic bases, and their application in topical cosmetic and pharmaceutical compositions that provide synergistic treatment and regulation of topical disorders of skin such as skin aging, wrinkles, acne, rosacea, age-spots, canker sores, striae distensae (stretch marks), pimples, and skin redness. Some of such in-situ prepared niacin derivatives do not show flushing effects (a warm feeling in the skin usually associated with redness and itching). Moreover, such derivatives of HA can be made in a stable topical formulation by the in-situ method from readily available starting materials. The in-situ method also permits the preparation of certain novel derivatives of HA with skin beneficial organic bases. The compositions made by the in-situ method possess the additional advantage that they can be made in anhydrous systems, solutions, or traditional water and oil emulsion systems, thus offering a wide choice of delivery systems.

### DETAILED DESCRIPTION OF THE INVENTION

The combination of HA with organic hetero-atom bases to form HA derivatives of such organic bases in a simple one step in-situ process is not known in the prior art. Surprisingly, such derivatives of HA with organic hetero-atom bases have been found to possess beneficial properties in the present invention that includes synergistic treatment and regulation of topical disorders of skin such as skin aging, wrinkles, acne, rosacea, age-spots, canker sores, striae distensae (stretch marks), pimples, and skin redness. Moreover, the pH of the formulation is not too low or too high for skin's compatibility. Most surprisingly, the AHA's or BHA's, in combination with organic hetero-atom bases have a more skin compatible pH and they have not been rendered ineffective, contrary to as mentioned above for the AHA's and BHA's that have been neutralized with alkali metal oxides, hydroxides, or ammonium hydroxide to increase their pH.

I have discovered a simple method by which derivatives of HA with organic hetero-atom bases (also referred to as “organic base” henceforth) can be made in-situ for their inclusion in cosmetic or pharmaceutical compositions that are useful for the synergistic treatment and regulation of topical disorders of skin aging, wrinkles, acne, rosacea, age-spots, canker sores, striae distensae (stretch marks), pimples, skin redness, and dry skin conditions of cracking, flaking, and scaling. The in-situ method comprises the mixing of HA with a suitable skin beneficial organic hetero-atom base in equimolar amounts in water, a combination of water and water-miscible organic solvent, or water-miscible organic solvent solution. The pH of such solutions, if formulated in compositions that contain water, is adjusted to specified limits to assure that the derivatization (complex formation) of HA with the organic base is complete. Any pH that is too high or too low from the optimum pH range can disrupt the derivatization of HA with the organic base. The optimum pH range is specific for each specific derivative. The optimum pH range for each specific derivative of HA with the organic base is determined by first preparing a solution of such derivative by an in-situ method in water or a mixture of water and water-miscible organic solvent, and then determining the pH of such solution of the derivative.

Although not bound by any theory, an explanation can be offered as to why the derivatives of HA reported in the present invention provide enhanced, synergistic efficacy, while the HA derivatives prepared from alkali metal oxides, hydroxides, ammonium hydroxide, or ethanol amines have poor bioavailability and essentially not effective in providing skin beneficial effects. It is theorized herein that the derivatives of HA with an organic base should not have a pH higher than the physiological pH of human body. The physiological pH of the human body is typically 7.4. Under certain conditions of exercise or stress, the physiological pH can drop to as low as 6.8. Thus, the pH range of 6.8 to 7.4 is considered a safe range for human physiological conditions. It is theorized that the derivatives of HA should have a pH so they can ionize and separate from each other once such derivatives have penetrated the upper layers of skin. This dissociation is important for the two ingredients that were initially combined to form HA derivative. After this dissociation, the two ingredients go their separate ways to provide their skin benefits. For example, sodium lactate has a pH of 9.5. It is poorly absorbed through the upper layers of skin in the first place, and after it enters the deeper layers of skin it still does not dissociate into lactic acid and sodium hydroxide, which are the two components that are used to make sodium lactate. On the other hand, the pH of niacinamide lactate is 3.6. This pH is much lower than the pH of lactic acid itself, which is 2.3, and hence it does not cause any irritation to skin compared to the irritation caused by lactic acid itself. When niacinamide lactate enters the deeper layers of skin, it dissociates easily at the physiological pH of the human body. This is because the physiological pH of human body at 7.4 is considered more alkaline than the pH of niacinamide lactate at 3.6. Additionally, the pH of niacinamide itself is 6.3, which is lower than the physiological pH of human body. Because of these pH differentials under the physiological pH conditions, Niacinamide and lactic acid moieties separate from niacinamide lactate molecule, and provide their skin rejuvenation properties when they are exposed to physiological pH conditions in the deeper layers of skin. These skin beneficial properties appear to be enhanced by a synergistic mechanism, which is due to greater bioavailability of niacinamide and lactic acid moieties from niacinamide lactate molecule, compared to the application of either niacinamide or lactic acid separate from each other in a skin care composition. This can be further explained by considering the composition of the physiological buffering system of human body. By far the most important buffer for maintaining acid-base balance in the blood is the carbonic-acid-bicarbonate buffer. The simultaneous equilibrium reactions of interest are shown below. It is clear to see that any organic base that

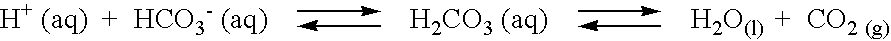

has pH higher than the carbonate anion will not dissociate easily from any derivative made with an HA. These physiological pH effects are further explained by Vander et al. (Human Physiology, 6th ed. WCB McGraw-Hill, Boston, 1994, p. 463-466, 492-3, 552-6).

However, the pH of the composition is not important for the preparation of any anhydrous delivery systems. This is due to the fact that derivatives of HA with organic bases do not usually ionize in anhydrous systems. Still, it must be noted here that such anhydrous systems must contain organic bases that do not have pH higher than the bicarbonate anion due to the reasons of their dissociation in the deeper layers of skin, as noted above.

As is known in the art, the union of an acid and base leads to the formation of a salt as part of a neutralization reaction. In the case of diacid and triacid bases, and of dibasic and tribasic acids, the mutual neutralization may vary in degree, producing respectively basic, neutral, or acid salts. A method for synthesizing water-soluble, single component, or multi-component salts of HA has now been discovered, which includes, for example, reacting ascorbic acid in water with at least one organic base to form a single component salt, or several organic bases to form a multi-component salt, the quantity of organic base or bases depending upon the molecular weight and acidity of organic base or bases to form salts with HA. While the preparation of such salts is not difficult, as set forth in U.S. patent application No. 20020058704 to Malik et al., the preparation of such salts of ascorbic acid, in water solution, that are stable in the presence of water and air, has been difficult in the prior art. The salts of HA with organic bases are called the “derivatives of HA with organic bases” henceforth. This is because such salts are specific chemical entities with their independent physical, chemical, and biological properties. The term “salts” is confusing to many consumers, as they relate this word to common salt, or sodium chloride. Of course, the derivatives of HA with organic bases, although technically they are salts, they are not the same as sodium chloride. This is an important aspect that requires careful attention when formulating compositions for the consumer markets.

To illustrate the scope of this invention, the equation 1 shows the formation of niacinamide salicylate, a derivative salicylic acid (a BHA) with niacinamide (an organic hetero-atom base), in water solution;

Salicylic Acid+Niacinamide→Niacinamide Salicylate (Equation 1)

Similarly, by mixing lactic acid with allantoin in equimolar amounts in water solution, one mole of allantoin lactate is produced in-situ, as illustrated in Equation 2.

Lactic Acid+allantoin→Allantoin Lactate (Equation 2)

Additionally, by mixing an inorganic acid salt of niacinamide or niacin with a metal salt of an organic acid, niacinamide or niacin derivatives of organic acids can be prepared in-situ, as depicted in Equation 3. This example also illustrates a method by which any HA derivative, such as sodium glycolate (which is essentially ineffective for skin beneficial applications, as mentioned above) can be converted into a skin beneficial HA derivative of an organic base.

Sodium Glycolate+Niacin hydrochloride→Niacin Glycolate+Sodium Chloride (Equation 3).

Multi-component derivatives of HA with organic bases can also be made by the in-situ method by mixing the reacting components in proportionate molar quantities in water or a mixture of water and water-miscible organic solvent solution, as illustrated in Equation 4.

Glycolic Acid+Hydroxycitric Acid+Ascorbic Acid+Salicylic Acid+Creatine→Creatine Glycolate+Creatine Hydroxycitrate+Creatine Ascorbate+Creatine Salicylate (Equation 4)

Novel derivatives of HA with skin beneficial organic bases can be made by in-situ method of present invention, as illustrated for the preparation of Glucosamine Ascorbyl Phosphate in Equation 5. This example also shows that sodium lactate, which is a poorly bioavailable derivative of lactic acid, can be converted into glucosamine lactate, which is a highly bioavailable derivative of lactic acid. In addition, while the sodium part of sodium lactate does not provide any skin beneficial effects, the glucosamine part of glucosamine lactate does provide collagen synthesis enhancement benefits of glucosamine, which results in the overall enhanced skin beneficial effects of lactic acid.

Sodium Ascorbyl Phosphate+Sodium Lactate+Glucosamine Hydrochloride=→Glucosamine Ascorbyl Phosphate+Glucosamine Lactate+Sodium Chloride (Equation 5)

Multi-component compositions of both previously unknown derivatives and previously known derivatives HA with organic bases can also be made, as illustrated in Equation 6.

Sodium Ascorbyl Phosphate+Lactic Acid+Glycolic Acid+Salicylic Acid+Glucosamine Hydrochloride+Yohimbine→Glucosamine Ascorbyl Phosphate+Yohimbine Lactate+Yohimbine Glycolate+Yohimbine Salicylate+Sodium Chloride (Equation 6)

The compositions in Equation 1 to 6 can also be made in anhydrous systems by using appropriate water-soluble organic solvent in place of water in the in-situ method. The water-miscible organic solvents include but not limited to glycerin, propylene glycol, butylene glycol, polyethylene glycol, polypropylene glycol, methyl pyrrolidone, pyrrolidone, butylene glycol, hexylene glycol, methylpropanediol, glycol ethers, ethanol, isopropanol, and such. A combination of water and water-miscible organic solvent can also be used for the preparation of HA derivatives with organic bases. The examples shown in Equation 1 to Equation 6 are only illustrative, and they do not represent any limitations of the scope of present invention

Although a great number of organic heteroatom bases are available, the selection of an appropriate organic base is made on the basis of the following criteria,

(i) the organic base should have a desirable and complementary skin beneficial effect synergistic to the HA moiety of the HA-organic base derivative,

(ii) the organic base should have a pH less than the physiological pH of human body, when such base is in combination with an HA,

(iii) the organic base should form a stable derivative in combination with an HA.

To illustrate the selection criteria for an organic base further, niacinamide is a good example. SaNogueira et al. (U.S. Pat. No. 6,174,533) discuss that while a variety of compounds have been described in the art as being useful for regulating fine lines, wrinkles, acne, pimples, and other forms of undesirable skin surface texture, niacinamide and niacin have shown most promise in regulating skin conditions including fine lines, wrinkles, uneven or rough surface, and photo-damaged skin. Niacinamide thus possess skin beneficial properties complementary to HA. The pH of niacinamide in water solution is 6.3, which is highly desirable since it is less than the physiological pH of 7.4. Niacinamide forms stable derivatives with most HA. The niacinamide-HA derivatives thus formed dissociate sufficiently under the conditions of physiological pH. The niacinamide-HA derivatives are formulatable in most cosmetic formulations.

In another example, glucosamine is a heteroatom base with many known skin beneficial effects. U.S. Pat. No. 6,440,465 (Meisner et al.) report the use of glucosamine in the treatment of psoriasis and other skin disorders. Although the mechanism of action of glucosamine is not well understood, it was shown almost 30 years ago that, in vitro, it significantly increases secretion of mucopolysaccharides by fibroblasts (N-acetylglucosamine and N-acetyl-galactosamine also worked, but to a lesser degree) Karzel K. and Domenjoz R., “Effects of hexosamine derivatives and uronic acid derivatives on glycosaminoglycans metabolism of fibroblast cultures,” Pharmacology 5: 337-345 (1971). This contrasts with the effects of steroids and non-steroidal anti-inflammatory drugs, which inhibit mucopolysaccharide metabolism by fibroblasts in vitro (and also appear to decrease connective tissue in vivo). Thus glucosamine, though anti-inflammatory, does not compromise normal connective tissue as do other anti-inflammatory agents. Glucosamine may work by inhibiting T-cell access to the skin as a result of the increased density of the connective tissue promoted by glucosamine. Glucosamine is thus complementary to HA in skin beneficial effects. The pH, formulation, and HA-derivative stability issues are all acceptable for glucosamine-HA derivatives. Mammone et al. (U.S. Pat. No. 6,413,525) disclose the surprising use of glucosamine derivatives for the exfoliation of skin. This benefit is highly complementary to AHA and BHA that are also known to provide exfoliating and keratolytic benefits for anti-aging compositions. In a disclosure, Bath et al. (U.S. Pat. No. 6,333,304) teach that glucosamine, an amino sugar, is a major constituent of hyaluronic acid and is preferentially taken up by chondrocytes and used in the synthesis of hyaluronic acid. By increasing the amount of hyaluronic acid, glucosamine supplementation leads to the rehydration of cartilage, resulting in increased lubrication and shock absorbing capability. Glucosamine supplementation also leads to an increase in proteoglycans in the extra cellular matrix of articular cartilage, thereby increasing the overall amount and the structural integrity of the cartilage. Glucosamine can thus hydrate the dermis and provide anti-aging benefits, complementary to several HA. Additional benefits of glucosamine are described by de los Reyes et al. (Progress in Drug Research, Vol. 55, page 84, 2000).

Allantoin is yet another example of an organic heteroatom base that provides skin beneficial effects complementary to several HA. U.S. Pat. No. 5,885,581 (Massand) claim the use of allantoin for the treatment of scar tissues. Farber et al. (U.S. Pat. No. 6,329,413) claim the use of allantoin in skin protectant compositions. Song et al. (U.S. Pat. No. 5,843,998) disclose the anti-inflammatory applications of allantoin in skin soothing compositions. Schulman (U.S. Pat. No. 5,503,822) use allantoin for preventing the formation of lesions and aphthous ulcers.

The skin beneficial HA derivative made from the combination of HA and organic base ingredients (indicated in the parentheses) prepared by the in-situ method, include but not limited to the following examples. Allantoin lactate (allantoin and lactic acid), allantoin glycolate (allantoin and glycolic acid), allantoin mandelate (allantoin and mandelic acid), allantoin malate (allantoin and malic acid), allantoin ascorbate (allantoin and ascorbic acid), allantoin phytate (allantoin and phytic acid), allantoin citrate (allantoin and citric acid), allantoin hydroxy citrate (allantoin and hydroxy citric acid), allantoin aleurate (allantoin and aleuritic acid), allantoin salicylate (allantoin and salicylic acid), allantoin hyaluronate (allantoin and hyaluronic acid), glucosamine lactate (glucosamine and lactic acid), glucosamine glycolate (glucosamine and glycolic acid0, glucosamine malate (glucosamine and malic acid), glucosamine mandelate (glucosamine and mandelic acid), glucosamine ascorbate (glucosamine and ascorbic acid, glucosamine phytate (glucosamine and phytic acid), glucosamine citrate (glucosamine and citric acid), glucosamine hydroxy citrate (glucosamine and hydroxy citric acid), glucosamine aleurate (glucosamine and aleuritic acid), glucosamine salicylate (glucosamine and salicylic acid), glucosamine hyaluronate (glucosamine and hyaluronic acid), creatine lactate (creatine and lactic acid), creatine glycolate (creatine and glycolic acid), creatine malate (creatine and malic acid), creatine mandelate (creatine and mandelic acid), creatine ascorbate (creatine and ascorbic acid), creatine phytate (creatine and phytic acid), creatine citrate (creatine and citric acid), creatine hydroxy citrate (creatine and hydroxy citric acid), creatine aleurate (creatine and aleuritic acid), creatine salicylate (creatine and salicylic acid), creatine hyaluronate (creatine and hyaluronic acid), niacinamide lactate (niacinamide and lactic acid), niacinamide glycolate (niacinamide and glycolic acid, niacinamide malate (niacinamide and malic acid), niacinamide mandelate (niacinamide and mandelic acid), niacinamide ascorbate (niacinamide and ascorbic acid), niacinamide phytate (niacinamide and phytic acid), niacinamide citrate (niacinamide and citric acid), niacinamide hydroxy citrate (niacinamide and hydroxy citric acid), niacinamide aleurate (niacinamide and aleuritic acid), niacinamide salicylate (niacinamide and salicylic acid), niacinamide hyaluronate (niacinamide and hyaluronic acid), pyridoxine lactate (pyridoxine and lactic acid), pyridoxine glycolate (pyridoxine and glycolic acid), pyridoxine malate (pyridoxine and malic acid), pyridoxine mandelate (pyridoxine and mandelic acid), pyridoxine ascorbate (pyridoxine and ascorbic acid, pyridoxine phytate (pyridoxine and phytic acid, pyridoxine citrate (pyridoxine and citric acid), pyridoxine hydroxy citrate (pyridoxine and hydroxy citric acid), pyridoxine aleurate (pyridoxine and aleuritic acid), pyridoxine salicylate (pyridoxine and salicylic acid), pyridoxine hyaluronate (pyridoxine and hyaluronic acid), chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate, and combinations thereof.

From the above examples, it is thus clear that the selection of a suitable organic base is important for its combination with an HA to form an HA derivative with most desirable complementary skin beneficial attributes.

The amount of skin HA derivatives of organic bases in the present invention is from about 0.1% to about 50% by weight, preferably from 5% to 20% by weight, most preferably from 1% to 10% by weight. A particular advantage of the current invention is that relatively large amounts of HA derivatives, up to about 50% by weight, can be incorporated in the formulation. If the amount of such derivatives is in excess of 50%, the crystallization may become a problem. However, the derivatives of HA with organic bases can be made in certain organic solvents in amounts higher than 50%. The skin care benefits of such compositions in such high concentrations are not known at this time, and thus not claimed in the present invention.

Although a great number of HA derivatives of organic bases are possible, the selection of an appropriate HA derivative is made on the basis of the following criteria,

(i) the organic base derivative of HA should be able to dissociate into the organic base and HA moieties under the conditions of physiological pH, when absorbed into the skin. This criterion is determined by the pH of the HA derivative in water (that pH should be lower than the physiological pH of human body of 7.4), and,

(ii) the organic base derivative of HA should be formulatable in cosmetic compositions to provide stable compositions.

The amount of water in the formulation is from about 0% to about 90%, preferably from about 10% to 60%. This is because the compositions that contain derivatives of HA of the present invention can be made in a variety of delivery systems that includes traditional water and oil emulsions, suspensions, colloids, liposomes, solutions, or anhydrous systems. The water can also come from the composition of the delivery system used in the present invention. For anhydrous systems, the water is typically much less than 1%. The present invention thus permits the formulation of a wide variety of compositions that can contain water or be anhydrous systems. Anhydrous systems may be preferred for certain applications, such as the preparation of high potency facial serums and skin whitening lotions, as will become clearer in the Examples section of this invention, whereas water and oil emulsions and suspensions are typically preferred for lotion, cream, gel, paste, and such.

The amount of the cosmetically acceptable delivery system in the formulation is from 1% to 80%, preferably from 10% to 60% by weight. The delivery system can comprise a base for lotion, cream, shampoo, serum, gel, salve, paste, spray, collodion, and such. The delivery system can be composed of one or more ingredients to provide skin elegance, skin feel, and enhanced bioavailability attributes popularly desired by the consumers.

The pH of the formulation is from about 3.0 to about 7.5, preferably from about 3.5 to about 6.5. The preferable pH is determined by the optimum stability of the complex that is derived from the combination of HA with appropriate skin beneficial organic base. As an illustration, the pH of the desired compositions in column 3 was determined from combining, in equimolar amounts, HA in column 1, with appropriate organic base in column 2, to give desired HA derivative in column 3. This preparation was done in-situ in a deionized water solution by mixing 0.01 mole of HA in 50 grams of deionized water and then determining the pH of the resulting solution, them preparing a solution or suspension of 0.01 mole of an organic base in column 2 in 50 grams of deionized water and determining its pH. The solution of HA in water obtained in column 1 is then combined with the solution of organic base in water obtained in column 2, to provide a solution of HA derivative of organic base in deionized water as per column 3. The pH of HA derivative thus obtained is also indicated in column 3. The pH in column 3 was determined to be optimal for any formulations that contained the HA-Organic Base derivative produced in column 3. This methodology can be followed for any other combinations of HA and organic bases.

| Column 1 | Column 2 | Column 3 |
|---|---|---|
| HA Acid Used | Organic Base Used | HA-Organic Base Derivative Formed |
| Ascorbic acid (pH 2.6) | Niacinamide (pH 6.3) | Niacinamide ascorbate (pH 3.7) |
| Salicylic acid (pH 2.5) | Niacinamide (pH 6.3) | Niacinamide salicylate (pH 3.5) |
| Lactic acid (pH 2.3) | Niacinamide (pH 6.3) | Niacinamide lactate (pH 3.6) |
| Mandelic acid (pH 2.2) | Niacinamide (pH 6.3) | Niacinamide mandelate (pH 3.3) |
| Sodium Lactate (pH 6.8) | Pyridoxine HCl (pH 2.8) | Pyridoxine Lactate (pH 4.2) |
| Sodium Hyaluronate (pH) | Pyridoxine HCl (pH) | Pyridoxine Hyaluronate (pH) |
| Lactic Acid (pH 2.3) | Allantoin (pH 3.9) | Allantoin Lactate (pH 2.5) |
| Sodium Lactate (pH 6.8) | Glucosamine HCl (pH 4.5) | Glucosamine Lactate (pH 5.6) |

If a surfactant is desired, then the amount of surfactant in the formulation is from 1% to 30%, preferably from 10% to 30% by weight. It is possible that the amount of surfactant in the formulation can be up to 40% by weight, but concentrations of surfactant greater than 30% increase the risk that the surfactant may gel out. Less than 10% by weight of surfactant is acceptable, but the foaming properties of the formulation are not as good for certain applications, such as a facial acne cleanser. Examples of surfactants that can be utilized are anionic, amphoteric, nonionic and cationic surfactants. Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic non-soap surfactants are, without limitation, the alkali metal salts of organic sulfate having in their molecular structure an alkyl radical containing from about 8 to about 22 carbon atoms and a sulfonic acid or sulfuric acid ester radical. Examples of Zwitterionic surfactants are, without limitation, derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxyl, sulfonate, sulfate, phosphate, or phosphonate. Examples of amphoteric surfactants are, without limitation, derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxyl, sulfonate, sulfate, phosphate, or phosphonate. Examples of cationic surfactants are, without limitation, stearyldimethylbenzyl ammonium chloride; dodecyltrimethyl ammonium chloride; nonylbenzylethyldimethyl ammonium nitrate; and tetradecylpyridinium bromide. Nonionic surfactants include, without limitation, compounds produced by the condensation of alkylene oxide groups (hydrophilic in nature) with an organic hydrophobic compound, which may be aliphatic or alkyl aromatic in nature, for example, the polyethylene oxide condensates of alkyl phenols.

Additional skin, hair, and body beneficial ingredients, such as other anti-aging ingredients, vitamins, hormones, analgesics, anesthetics, sun screens, skin whiteners, anti-acne agents, anti-bacterial agents, anti-fungal agents, botanical extracts, pharmaceuticals, processing-aids, minerals, plant extracts, concentrates of plant extracts, emollients, moisturizers, skin protectants, humectants, silicones, skin soothing ingredients, colorants, perfumes, and like can be added to the formulation. The quantities of such ingredients can be as needed, and not limited to any specific limits.

It is also common to use rheology modifiers for the control of viscosity and to provide skin feel attributes in cosmetic compositions. A variety of rheology modifiers can be used in the compositions of the present invention. The examples of rheology modifiers include, without limitation, Aristoflex AVC (Ammonium Acryloyldimethyltaurate/VP Copolymer), Structure Plus and Structure XL (Acrylates/Aminoacrylates/C 10-30 Alkyl PEG-20 Itaconate Copolymer), Carbomer, Xanthan Gum, Gellan Gum, Gum Arabic, Bentonite, various Clays, Silicas, Fumed Silica, Zeolites, Carbopol ETD 2020 (Acrylate C10-30 Alkyl Acrylate Crosspolymer), Rheocin (trihydroxystearin), Hydramol PGDS (PEG-90 Diisostearate), C24-28 Alkyl Dimethicone, Behenyl alcohol, and other similar materials.

The teachings of the present invention also permit the preparation of improved pharmaceutical compositions. For example, salicylic acid is a known drug ingredient approved by the FDA (Food & Drug Administration) for the cure of acne in the USA. However, if two such formulations from two different competing manufacturers are each made with, let us say 2% salicylic acid, then the clinical efficacy of these two formulations is expected to be very similar. However, by also using only 1% to 2% of niacinamide salicylate, prepared as described in the present invention, in combination with 2% salicylic acid, the clinical efficacy for the cure of acne can now be improved over the formulations that contain 2% salicylic acid only. Similarly, hydroquinone is a drug approved by the FDA for skin whitening compositions. Again, if two competing products had the same amount of hydroquinone, let us say 2%, then the skin whitening benefit will be expected to be same for these two products. However, by also including only 1% to 2% of yohimbine ascorbate in one of these two formulations the skin whitening properties are significantly enhanced, in comparison to the formulation that contains only hydroquinone.

The following terms used herein have the meanings set forth below.

Acidity of Organic Base. The combining power of an organic base with reference to an acid.

Alkaloid. Any of a class of nitrogenous organic bases, especially one of a vegetable origin, having a physiological effect on animals and man, as morphine.

Amino Acid. Any of a group of organic compounds containing the amino group combined with the carboxyl radical.

Amino Ester. Any of a group of organic compounds containing an amino group combined with the carboxyl radical that has been reacted with an alcohol radical to form an ester radical.

Base. A compound that is capable of so uniting with an acid as to neutralize it and form a salt.

Basic. A compound that has base-like properties.

Derivative. A compound formed or regarded as being formed from a specified substance or another compound, usually by partial substitution.

Dispersion. An emulsion or suspension. Comprise the dispersed substance and the medium it is dispersed in.

Emulsion. Intimate mixture of two incompletely miscible liquids.

Equimolar. Of equivalent molecular weight.

Heteroatom Base. A base that contains a heteroatom for its base-like property, such as a nitrogen atom. This excludes bases such as sodium hydroxide, potassium hydroxide, etc. since such bases are not organic in nature and they also do not contain a base-like heteroatom in their molecule. This also excludes ammonium hydroxide, since that is not organic in nature.

Hydrophilic. Strong affinity for water.

Hydrophobic. Weak affinity for water.

Lipophilic. Strong affinity for fats or other lipids.

Miscible. Capable of mixing in any ratio without separation of the two phases. The mixture formed by a miscible liquid or solid is a solution.

Oleophilic. Strong affinity for oils.

Organic. Being, containing, or relating to carbon compounds, especially in which hydrogen is attached to carbon whether derived from living organisms or not.

Organic Base. An organic compound that also contains one or more nitrogen atoms that can bind with an organic or inorganic acid to form a salt.

Organic solvent. A solvent including a carbon compound. Examples include, without limitation, glycerin, PEG-6 (Polyethylene glycol 300), and Methylpropanediol.

Salt. Any compound consisting of the cation of a base and the anion of an acid, combined in proportions that give a balance of electropositive and electronegative charges.

Signs of Skin Aging. These include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adrenal structures such as sweat gland ducts, sebaceous glands, or hair follicles), or unevenness or roughness, loss of skin elasticity (loss and/or inactivation of functional skin elastin), sagging (including loss and/or damage to functional subcutaneous muscle tissue and including puffiness in the eye area and jowls), loss of skin firmness, loss of skin tightness, loss of skin recoil from deformation, discoloration (including under eye circles), blotching, shallowness, hyper pigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

Solution. A solid, liquid, or gas mixed homogeneously with a liquid.

Solvent. A substance capable of or used in dissolving or dispersing one or more other substances, especially a liquid component of a solution present in greater amount than the solute.

Suspension. Particles mixed in a fluid or a solid, but undissolved.

Synergism. The joint action of different substances in producing an effect greater than the sum of effects of all the substances acting separately.

Synergistic. Acting together

Water miscible organic solvent. An organic solvent that can be mixed with water in any ratio without separation of the water from the organic solvent. In the practice of the invention, the preferred (but not required) water miscible organic solvents are those commonly used in cosmetic applications, for example, glycerin, ethylene glycol, propylene glycol, butylene glycol, hexylene glycol, pyrrolidone, N-methyl pyrrolidone, dimethyl sulfoxide, dimethyl sulfone, polyethylene glycol, polypropylene glycol, methylpropanediol, and similar solvents.

### EXAMPLES

The following examples are presented to illustrate presently preferred practice thereof. As illustrations they are not intended to limit the scope of the invention. The examples illustrate both the in-situ preparation of HA derivatives of organic bases and their application in skin beneficial cosmetic and pharmaceutical compositions. All concentrations are in weight %.

### Example 1

### The In-Situ Preparation of Glucosamine Ascorbate

| Ingredients | Column 1 | Column 2 |
|---|---|---|
| Deionized Water | to | to |
| | 100 | 100 |
| Glucosamine Hydrochloride | 21.5 | 0.0 |
| Sodium Ascorbate | 19.8 | 0.0 |
| Propylene Glycol | 25.0 | 25.0 |
| Glucosamine Ascorbate | 0.0 | 35.5 |
| Geogard 221 (preservative) | 0.5 | 0.5 |
| Sodium Chloride | 0.0 | 5.85 |

Procedure: All ingredients in Column 1 were mixed and heated at 40 to 50 C for 30 minutes. The product was cooled. A thin solution of composition in Column 2 was obtained, pH 5.6. This ingredient can be stored for future use in cosmetic compositions.

### Example 2

This example shows the in-situ preparation of a total of 22% of niacinamide ascorbate and niacinamide phytate, and their utilization in a facial cleanser skin whitening, anti-aging composition. Column 1 describes the ingredients as they are used in the formulation. Column 2 describes the final composition resulting from the in-situ formation of niacinamide ascorbate and niacinamide phytate. There is more water in column 2, as the water contained in phytic acid solution is added to water in column 1. All six phosphoric acid groups are bound with niacinamide in column 2.

| Ingredient | Column 1 | Column 2 |
|---|---|---|
| Glycerin (water miscible organic solvent) | to | to |
| | 100 | 100 |
| Methyl paraben (preservative) | 0.2 | 0.2 |
| Ascorbic Acid | 9.0 | 0.0 |
| Phytic Acid (50% solution) | 6.6 | 0.0 |
| Niacinamide (nicotinamide) | 9.66 | 0.0 |
| Niacinamide Ascorbate | 0.0 | 15.0 |
| Niacinamide Phytate | 0.0 | 6.96 |
| Water | 15.0 | 18.3 |
| Phenoxyethanol (preservative) | 0.9 | 0.9 |
| Tauranol I-78-6 (Sodium Cocoyl | 20.0 | 20.0 |
| Isethionate) (surfactant) | | |
| Tauranol ws conc. (Sodium Methyl | 5.0 | 5.0 |
| Cocoyl Taurate) (surfactant) | | |
| Actiplex 2789 (Extract of various plants) | 0.1 | 0.1 |
| Fragrance | 0.5 | 0.5 |

Procedure: Mix water, ascorbic acid, and phytic acid in a tank separately. All of the other ingredients are then added, and the mixture is heated and stirred at 60 to 70 degrees C. for about five to ten minutes until the mixture is homogenous. The homogeneous mixture is cooled to room temperature. A paste-like product is formed. The stabilized niacinamide ascorbate formulation is used as a facial, hair, and body cleanser. It should be noted that when the composition is first mixed, as shown in Column 1, it is white in color. After preparation of the batch is complete, the product turns bright yellow, indicating the formation of niacinamide ascorbate, which is naturally yellow in color. The color meter readings were L 88.94, a 7.21, b 25.20.

### Example 3

The paste of Example I is stored at room temperature in a sealed container in the presence of air. After six months the paste is still yellow. A colorimetric reading with a color meter, such as Hunter Color Meter, shows that the color reading has changed by only 5%, and the product is still stable, and has not separated into solid and liquid phases. The color meter readings were L 86.43, a—7.5, b 24.46.

### Example 4

### Anti-Acne and Anti-Rosacea Face Mask with In-Situ Preparation of Chitosan Lactate, Chitosan Glycolate, Chitosan Salicylate, Chitosan Ascorbate, and Chitosan Mandelate

| Ingredients | Column 1 | Column 2 |
|---|---|---|
| Deionized Water | to | to |
| | 100 | 100 |
| Chitosan | 8.0 | 0.0 |
| Glycerin | 5.0 | 5.0 |
| Geogard 221 (Preservative) | 0.5 | 0.5 |
| Lactic Acid | 0.9 | 0.0 |
| Salicylic Acid | 1.38 | 0.0 |
| Glycolic Acid | 0.76 | 0.0 |
| Mandelic Acid | 1.52 | 0.0 |
| Ascorbic Acid | 1.76 | 0.0 |
| Chitosan Lactate | 0.0 | 2.5 |
| Chitosan Salicylate | 0.0 | 3.0 |
| Chitosan Glycolate | 0.0 | 2.3 |
| Chitosan Mandelate | 0.0 | 3.1 |
| Chitosan Ascorbate | 0.0 | 3.3 |

Procedure: All ingredients in Column 1 were mixed and heated at 40 to 50 C for 30 minutes. The mixture was cooled to room temperature. A clear gel was obtained, with analysis reported in Column 2.

### Example 5

### The In-Situ Preparation of a 36.5% High Potency Benzyl Niacin Mandelate Anti-Wrinkle Serum from Benzyl Niacin and Mandelic Acid.

| Ingredients | Column 1 | Column 2 |
|---|---|---|
| Mandelic Acid | 15.2 | 0.0 |
| Benzyl Niacin | 21.3 | 0.0 |
| Deionized Water | 30.0 | 30.0 |
| Propylene Glycol | 34.5 | 34.5 |
| Benzyl Niacin Mandelate | 0.0 | 36.5 |

Procedure: All ingredients in Column 1 were mixed and heated at 40 to 50 C for 30 minutes. The product was cooled. A thin solution of composition in Column 2 was obtained.

### Example 6

### Preparation of an Eye, Face, and Neck Gel with Niacinamide Salicylate, Niacinamide Glycolate, and Other Skin Beneficial Ingredients.

| Ingredient | Column 1 | Column 2 |
|---|---|---|
| PEG-6 | to | to |
| | 100 | 100 |
| Aristoflex AVC | 1.00 | 1.00 |
| Glycerin USP | 5.00 | 5.00 |
| Water | 20.00 | 20.32 |
| Geogard 221 (preservative) | 0.50 | 0.50 |
| Vitamin E Acetate | 0.50 | 0.5 |
| Niacinamide | 2.44 | 0.00 |
| Glycolic Acid (70%) | 1.08 | 0.0 |
| Salicylic Acid | 1.38 | 0.0 |
| Dimethicone | 4.00 | 4.00 |
| Dimethiconol | 4.00 | 4.00 |
| Cetyl Dimethiconol | 2.00 | 2.00 |
| Niacinamide Glycolate | 0.0 | 1.98 |
| Niacinamide Salicylate | 0.0 | 2.6 |

Procedure: Mix all ingredients in Column 1 and heat at 60 to 70 C for 30 minutes. Cool to room temperature, and adjust pH to 4.5 with sodium hydroxide solution. A clear pale yellow gel of composition in Column 2 was obtained. The additional water in column 2 is from glycolic acid, which contains 30% water in it.

### Example 7

### Rosacea and Acne Treatment Gel

This example shows the in-situ preparation of an anti-acne and anti-rosacea composition that contains multi-component mixture of several derivatives of HA with niacinamide, an organic base. The composition also contains some free, underivatized HA to show the versatility of the in-situ preparation method. Column 1 describes the ingredients as they are used in the formulation. Column 2 describes the final composition resulting from the in-situ formation of HA-organic base derivatives.

| Ingredient | Column 1 | Column 2 |
|---|---|---|
| Glycerine | to | to |
| | 100 | 100 |
| Niacinamide | 3.66 | 0.0 |
| Deionized Water | 20.0 | 20.0 |
| Geogard 221 (Preservative) | 0.5 | 0.5 |
| Dow Corning 2501 Wax | 10.0 | 10.0 |
| Structure Plus | 4.0 | 4.00 |
| Eyebright Extract | 0.1 | 0.1 |
| Botanicals Extracts Blend | 0.1 | 0.1 |
| Vitamin E Acetate | 0.1 | 0.1 |
| Lactic Acid | 1.9 | 1.0 |
| N-Acetyl-Cysteine | 2.13 | 0.5 |
| Salicylic Acid | 2.38 | 1.0 |
| Niacinamide Lactate | 0.0 | 2.12 |
| Niacinamide N-Acetyl-Cystinate | 0.0 | 2.85 |
| Niacinamide Salicylate | 0.0 | 2.6 |

Procedure: All the ingredients in column 1 were mixed and heated at 60 to 70 C for 30 minutes. A yellow mixture was obtained. The composition of this mixture is shown in Column 2. The product is Column 2 was obtained as a light yellow gel, useful for face and neck zone anti-acne, anti-age and anti-wrinkle applications. The product has the following properties; pH 4.5.

### Example 8

This example illustrates the preparation of a skin whitening formula that contains hydroquinone as the drug active ingredient, with niacinamide lactate and lactic acid added to boost the skin-whitening efficacy of hydroquinone. Column 1 shows the ingredients as they are added in the formulation. Column 2 shows the final composition of the formulation.

| | Column 1 | Column 2 |
|---|---|---|
| 1. Carbowax 300 (PEG-6) | To | to |
| | 100 | 100 |
| 2. Aristoflex AVC | 0.8 | 0.8 |
| 3. Deionized Water | 15.0 | 15.0 |
| 4. Niacinamide | 1.22 | 0.0 |
| 5. Hydroquinone | 4.0 | 4.0 |
| 6. Jeechem HPIB (silicone blend) | 10.0 | 10.0 |
| 7. Killitol (preservative) | 0.3 | 0.3 |
| 8. Lactic Acid | 1.8 | 0.9 |
| 9. Niacinamide Lactate | 0.0 | 2.12 |

Process: Mix 2 and 3 till a clear gel is formed. Add 1, 8, and 9 and heat at 50 to 60 C. Add all other ingredients with mixing. Cool to room temperature. A translucent cream is obtained, pH 3.0. This product does not cause skin irritation, even at this low pH.

### Example 9

This Example Shows In-Situ Preparation of Benzyl Niacin Ascorbate, Benzyl Niacin Lactate, and Benzyl Niacin Mandelate and their formulation with an oil-soluble vitamin for an anti-acne, skin whitening, and anti-wrinkle composition. Column 1 shows the ingredients as they are added. Column 2 shows the final composition of the formulation.

| Ingredients | Column 1 | Column 2 |
|---|---|---|
| 1. Carbowax 300 | to | to |
| | 100 | 100 |
| 2. Aristoflex AVC | 1.0 | 1.0 |
| 3. Glycerin | 5.0 | 5.0 |
| 4. Deionized Water | 20.0 | 20.0 |
| 5. Vitamin E Acetate | 2.1 | 2.1 |
| 6. Geogard 221 (Preservative) | 0.5 | 0.5 |
| 7. Dimethicone | 4.0 | 4.0 |
| 8. Benzyl Niacin | 6.39 | 0.0 |
| 9. Ascorbic Acid | 1.76 | 0.0 |
| 10. Lactic Acid | 0.90 | 0.0 |
| 11. Mandelic Acid | 1.52 | 0.0 |
| 12. Dimethiconol | 4.0 | 4.0 |
| 13. Cetyl Dimethicone Copolyol | 2.0 | 2.0 |
| 14. Benzyl Niacin Ascorbate | 0.0 | 3.89 |
| 15. Benzyl Niacin Lactate | 0.0 | 3.03 |
| 16. Benzyl Niacin Mandelate | 0.0 | 3.65 |

Process: Mix 2, 3, and 4 till a clear gel is formed. Add all other ingredients and mix. Heat at 40 to 50 C for 30 minutes. Cool to room temperature. A translucent cream is obtained. It is rapidly absorbed upon application to skin. No skin flushing is observed. (Skin flushing is observed when only benzyl niacin is applied to skin.)

### Example 10

This example illustrates the preparation of an anhydrous 45.4% serum of methyl niacin lactate for a very high potency skin rejuvenating serum. Column 1 shows the ingredients as they are used in the formulation. Column 2 shows the final composition of the formulation.

| Ingredients | Column 1 | Column 2 |
|---|---|---|
| 1. Carbowax (PEG-6) | to | to |
| | 100 | 100 |
| 2. Methyl Niacin (methyl nicotinate) | 27.4 | 0.0 |
| 3. Lactic Acid | 18.0 | 0.0 |
| 4. Methyl Niacin Lactate | 0.0 | 45.4 |

Process: Mix all ingredients till a clear solution is obtained. The pH of this product is not important, as it does not contain any water (anhydrous composition).

### Example 1

This example illustrates the preparation of a facial acne treatment cream that contains salicylic acid as a drug active ingredient with the enhancement of its efficacy by the inclusion of niacinamide salicylate and niacinamide lactate prepared by the in-situ method of the present invention.

| Ingredients | Column 1 | Column 2 |
|---|---|---|
| 1. Polyethylene glycol (PEG-6) | to 100 | to 100 |
| 2. Aristoflex AVC | 1.0 | 1.0 |
| (ammonium acryloyldimethyltaurate/vp | | |
| copolymer) | | |
| 3. Deionized water | 15.0 | 15.0 |
| 4. Salicylic Acid | 3.38 | 2.0 |
| 5. Lactic Acid | 0.9 | 0.0 |
| 6. Niacinamide | 2.44 | 0.0 |
| 7. Killitol (preservative) | 0.3 | 0.3 |
| 8. Jeesilc 6056 (polydimethylsiloxane) | 10.0 | 10.0 |
| 9. Actiplex (plant extracts blend) | 0.1 | 0.1 |
| 10. Titanium dioxide | 0.2 | 0.2 |
| 11. Niacinamide Salicylate | 0.0 | 2.6 |
| 12. Niacinamide Lactate | 0.0 | 2.12 |

Procedure: Mix 2 and 3 till clear. Add all other ingredients except 9. Heat at 50 to 60 C for 30 minutes till all solids dissolved. Cool to room temperature with slow mixing. Add 9. Mix till homogeneous. A white cream is obtained.

### Example 12

A composition with allantoin lactate, allantoin glycolate, and allantoin mandelate useful for an eye-zone anti-wrinkle treatment. Column 1 shows the ingredients as they are added in the composition. Column 2 shows the final composition of the formulation.

| Ingredient | Column 1 | Column 2 |
|---|---|---|
| Deionized water | to 100 | to 100 |
| GMS-SE | 6.0 | 6.0 |
| Stearic Acid | 3.0 | 3.0 |
| Cetyl Alcohol | 4.0 | 4.0 |
| Glycerine | 1.0 | 1.0 |
| Jojoba Oil | 0.1 | 0.1 |
| Structure Plus | 2.0 | 2.0 |
| Glydant Plus Liquid (preservative) | 0.3 | 0.3 |
| Crodafose CES | 4.0 | 4.0 |
| Cyclomethicone | 4.0 | 4.0 |
| Tween-20 | 4.0 | 4.0 |
| Dimethicone | 2.0 | 2.0 |
| Vitamin E Acetate | 0.1 | 0.1 |
| Panthenol | 0.1 | 0.1 |
| Vitamin A Palmitate | 0.1 | 0.1 |
| Sweet Almond Oil | 0.2 | 0.2 |
| Sesame Oil | 0.2 | 0.2 |
| Apricot Kernel Oil | 0.2 | 0.2 |
| Mandelic Acid | 1.52 | 0.0 |
| Lactic Acid | 0.90 | 0.0 |
| Glycolic Acid (70%) | 1.08 | 0.0 |
| Allantoin | 4.74 | 0.0 |
| Allantoin Lactate | 0.0 | 2.48 |
| Allantoin Glycolate | 0.0 | 2.34 |
| Allantoin Mandelate | 0.0 | 3.1 |

Procedure: The HA's are dissolved in the deionized water to produce an aqueous solution of HA's. The remaining ingredients are mixed together in a separate tank and heated at 70 to 80 C till homogeneous to form supplemental mixture. The aqueous solution is added to the tank and admixed with the supplemental mixture to produce a homogeneous stabilized formulation

I claim:

1. A cosmetic or pharmaceutical composition for synergistic treatment or prevention of topical disorders of skin such as cracking, flaking, or scaling of the skin, acne, rosacea, skin wrinkles, age-spots, canker sores, striae distensae (stretch marks), pimples, and redness comprising:
(i) a skin beneficial Hydroxy Acid (HA) derivative, ranging from about 0.1% to about 50%, selected from a derivative of HA with at least one organic hetero-atom base,
(ii) from about 1% to about 90% of water,
(iii) from about 1% to about 99% of a cosmetically acceptable delivery system, and,
(iv) the pH of the composition from about 3.0 to about 7.5.

2. A composition according to claim 1 wherein the skin beneficial HA derivative is prepared by an in-situ method from a combination of an HA with an organic heteroatom base.

3. A composition according to claim 1 wherein the skin beneficial HA derivative is selected from allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate, and combinations thereof.

4. A composition according to claim 1 wherein the cosmetically acceptable delivery system is selected from a lotion, cream, shampoo, shower gel, cleanser, bath oil, salve, paste, lip balm, serum, gel, body splash, cologne, liposomes, mask, mud, and other such well known topical cosmetic and pharmaceutical delivery systems.

5. The compositions according to claim 1 wherein the cosmetically acceptable delivery system can be traditional water and oil emulsions, suspensions, solutions, gels, colloids, and anhydrous systems, and combinations thereof.

6. A composition according to claim 1 wherein additional skin beneficial ingredients, such as anti-oxidants, surfactants, cleansing agents, bleaching agents, vitamins, hormones, minerals, plant extracts, skin whitening agents, anti-inflammatory agents, concentrates of plant extracts, emollients, moisturizers, skin protectants, humectants, silicones, skin soothing ingredients, sun screens, analgesics, anesthetics, colorants, perfumes, and like can be added to the formulation. The quantities of such ingredients can be as needed, and not limited to any specific amounts.

7. A composition according to claim 2 wherein the skin beneficial HA is selected from Glycolic Acid, Malic Acid, Lactic Acid, Mandelic Acid, Ascorbic Acid, Phytic Acid, Salicylic Acid, Aleuritic Acid, Tartaric Acid, Citric Acid, Hydroxytetronic Acid, Glucuronic Acid, Hyaluronic Acid, Mucic Acid, Galacturonic Acid, Gluconic Acid, Saccharic Acid, Glucoheptonic Acid, alpha-Hydroxybutyric Acid, Tartronic Acid, alpha-Hydroxyisobutyric Acid, Isocitric Acid, alpha-Hydroxyisocaproic Acid, Dihydroxymaleic Acid, alpha-Hydroxyisovaleric Acid, Dihydroxytartaric Acid, beta-Hydroxybutyric Acid, Dihydroxyfumaric Acid, beta-Phenyllactic Acid, Atrolactic Acid, Galactonic Acid, Pantoic Acid, Glyceric Acid, and their derivatives, and combinations thereof.

8. A composition according to claim 2 wherein the skin beneficial organic heteroatom base is selected from cyclic or acyclic amines, amino acid esters, purines, pyrimidines, nucleic acids, nucleotides, DNA, RNA, peptide esters, guanidines, amidines, amino sugars, oxazoles, pyrroles, pyrazoles, imidazoles, pyridines, quinolines, quinazolines, quinoxalines, alkaloids, and their derivatives, and combinations thereof.

9. A composition according to claim 2 wherein the skin beneficial organic hetero-atom base is selected from allantoin, arginine esters, lysine esters, cysteine esters, cystine esters, DNA, RNA, Adenosine, Guanosine, Cytosine, niacinamide, methyl niacin, benzyl niacin, lauryl niacin, pyridoxine, pyridoxal, pyridoxamine, yohimbine, vincamine, vinpocetin, glucosamine, chitosan, chondroitin, glutathione, carnosine, creatine, creatinine and their derivatives, and combinations thereof.

10. A composition according to claim 2 wherein the skin beneficial organic hetero-atom base is selected from topical anesthetics that includes benzocaine, dibucaine, dyclonine, lidocaine, pramoxine, tetracaine, ephedrine, epinephrine, and phenylephrine, and combinations thereof.

11. A composition according to claim 2 wherein the skin beneficial organic hetero-atom base can be inorganic acid salts of said organic hetero-atom bases that can be selected from glucosamine sulfate, glucosamine hydrochloride, chondroitin sulfate, chondroitin hydrochloride, lysine ester hydrochloride, lysine ester sulfate, arginine ester hydrochloride, arginine ester sulfate, niacinamide hydrochloride, niacinamide sulfate, niacin hydrochloride, niacin sulfate, yohimbine hydrochloride, yohimbine sulfate, benzocaine hydrochloride, dibucaine hydrochloride, tetracaine hydrochloride, pramoxine hydrochloride, lidocaine hydrochloride, diclonine hydrochloride, ephedrine sulfate, epinephrine hydrochloride, and phenylephrine hydrochloride, chitosan hydrochloride, chitosan sulfate, and combinations thereof.

12. A composition according to claim 3 wherein skin beneficial HA derivative can also be written in terms of the HA and organic base ingredients from which such HA derivatives are prepared by the in-situ method, as indicated within the parentheses for HA derivatives according to claim 3. Allantoin lactate (allantoin and lactic acid), allantoin glycolate (allantoin and glycolic acid), allantoin mandelate (allantoin and mandelic acid), allantoin malate (allantoin and malic acid), allantoin ascorbate (allantoin and ascorbic acid), allantoin phytate (allantoin and phytic acid), allantoin citrate (allantoin and citric acid), allantoin hydroxy citrate (allantoin and hydroxy citric acid), allantoin aleurate (allantoin and aleuritic acid), allantoin salicylate (allantoin and salicylic acid), allantoin hyaluronate (allantoin and hyaluronic acid), glucosamine lactate (glucosamine and lactic acid), glucosamine glycolate (glucosamine and glycolic acid0, glucosamine malate (glucosamine and malic acid), glucosamine mandelate (glucosamine and mandelic acid), glucosamine ascorbate (glucosamine and ascorbic acid, glucosamine phytate (glucosamine and phytic acid), glucosamine citrate (glucosamine and citric acid), glucosamine hydroxy citrate (glucosamine and hydroxy citric acid), glucosamine aleurate (glucosamine and aleuritic acid), glucosamine salicylate (glucosamine and salicylic acid), glucosamine hyaluronate (glucosamine and hyaluronic acid), creatine lactate (creatine and lactic acid), creatine glycolate (creatine and glycolic acid), creatine malate (creatine and malic acid), creatine mandelate (creatine and mandelic acid), creatine ascorbate (creatine and ascorbic acid), creatine phytate (creatine and phytic acid), creatine citrate (creatine and citric acid), creatine hydroxy citrate (creatine and hydroxy citric acid), creatine aleurate (creatine and aleuritic acid), creatine salicylate (creatine and salicylic acid), creatine hyaluronate (creatine and hyaluronic acid), niacinamide lactate (niacinamide and lactic acid), niacinamide glycolate (niacinamide and glycolic acid, niacinamide malate (niacinamide and malic acid), niacinamide mandelate (niacinamide and mandelic acid), niacinamide ascorbate (niacinamide and ascorbic acid), niacinamide phytate (niacinamide and phytic acid), niacinamide citrate (niacinamide and citric acid), niacinamide hydroxy citrate (niacinamide and hydroxy citric acid), niacinamide aleurate (niacinamide and aleuritic acid), niacinamide salicylate (niacinamide and salicylic acid), niacinamide hyaluronate (niacinamide and hyaluronic acid), pyridoxine lactate (pyridoxine and lactic acid), pyridoxine glycolate (pyridoxine and glycolic acid), pyridoxine malate (pyridoxine and malic acid), pyridoxine mandelate (pyridoxine and mandelic acid), pyridoxine ascorbate (pyridoxine and ascorbic acid, pyridoxine phytate (pyridoxine and phytic acid, pyridoxine citrate (pyridoxine and citric acid), pyridoxine hydroxy citrate (pyridoxine and hydroxy citric acid), pyridoxine aleurate (pyridoxine and aleuritic acid), pyridoxine salicylate (pyridoxine and salicylic acid), pyridoxine hyaluronate (pyridoxine and hyaluronic acid), chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate and combinations thereof.
